(19) United States
(12) Patent Application Publication
(10) Pub. No.: US 2017/0290871 A1
(43) Pub. Date: Oct. 12, 2017

(54) METHOD OF TREATING HYPERTENSION

(71) Applicant: KING SAUD UNIVERSITY, RIYADH (SA)

(72) Inventors: AMANI SHAFEEK AWAAD, RIYADH (SA); GHADA MOHAMED ZAIN, RIYADH (SA); REHAM M. EL-MELIGY, RIYADH (SA); AMAL AHMED SAFHI, RIYADH (SA); VIDYA DEVENATHADESIKAN SESHADRI, RIYADH (SA); SHEKHAH SAUD ALMOQREN, RIYADH (SA); NOURAH AHMED AN QURAIN, RIYADH (SA)

(21) Appl. No.: 15/096,228

(22) Filed: Apr. 11, 2016

(51) Int. Cl. A61K 36/28; A61K 9/00

(52) U.S. Cl. CPC A61K 36/28; A61K 9/0053; A61K 2236/15; A61K 2236/17; A61K 2236/33; A61K 2236/51; A61K 2236/331; A61K 2236/53

(57) ABSTRACT

A method of treating hypertension can include administering to a patient in need thereof a therapeutically effective amount of an extract of Matricaria chamomilla L. The extract can be administered orally to the patient in an amount of about 100 mg/kg to about 200 mg/kg.

### BACKGROUND OF THE INVENTION

### 1. Field of the Invention

The present invention relates to the treatment of hypertension, and particularly to the treatment of hypertension using Matricaria chamomilla extracts.

### 2. Description of the Related Art

Hypertension is a common cause of cardiovascular disease. The prevalence varies with age, race, education and many other variables. Data from the National Health and Nutrition Examination Survey (NHANES) indicates that more than 50 million Americans have high blood pressure. As many as 1 billion individuals are afflicted with hypertension and approximately 7.1 million deaths per year may be attributable to hypertension. The World Health Organization (WHO), for example, reports that hypertension is responsible for 62% of cerebrovascular disease and 49% of ischemic heart disease with little variation by sex. Many synthetic drugs have been used for the treatment of hypertension due to the severity and occurrence of the disease. Most of these drugs, however, have numerous side effects.

Natural products and compounds derived from folk medicine have been gaining importance in health care not only because of less toxicity and side effects than pharmaceutical drugs, but also because of their role of quenching reactive oxygen species (ROS).

Thus, extracts of the plant Matricaria chamomilla as antihypertensive agents solving the aforementioned problems are desired.

### SUMMARY OF THE INVENTION

A method of treating hypertension can include administering to a patient in need thereof a therapeutically effective amount of an extract of Matricaria chamomilla. The extract can be administered orally to the patient in an amount of about 100 mg/kg to about 200 mg/kg. The extract can be prepared using alcohol extraction, water distillation (water distillation method), or water distillation and lyophilization (water distillation and lyophilization method). Preferably, the sample includes aerial parts of the Matricaria chamomilla plant, e.g., leaves and/or flowers.

These and other features of the present invention will become readily apparent upon further review of the following specification.

### BRIEF DESCRIPTION OF THE DRAWINGS

FIG. 1 is a graph showing the effect of a single dose of the Chamomile extracts on blood pressure (BP) of normotensive rats.

FIG. 2 is a graph showing the effect of a single dose of the Chamomile extracts on the heart rate (HR) of normotensive rats.

FIG. 3 is a graph showing the effect of the Chamomile extracts on blood pressure (BP) and heart rate (HR) of control and that of hypertension Induced Rats.

FIG. 4 is a graph showing the effect of oral administration of a single cup (250 mL) of chamomile beverage on the SBP and DBP & heart rate in normotensive human volunteers (n=50).

FIG. 5 is a graph showing the effect of oral administration of a single cup (250 mL) with one teaspoonful (1×˜5 g) of chamomile flower extract on the SBP and DBP in mildly hypertensive human volunteers (n=50).

FIG. 6 is a graph showing the effect of oral administration of a single cup (250 mL) with two teaspoonful (2×˜5 g) of chamomile flower on the SBP and DBP in mildly hypertensive human volunteers (n=50).

FIG. 7 is a graph showing the effect of oral administration of a single cup (250 mL) with three teaspoonful (3×˜5 g) of chamomile flower on the SBP and DBP in mildly hypertensive human volunteers (n=50).

Similar reference characters denote corresponding features consistently throughout the attached drawings.

### DETAILED DESCRIPTION OF THE PREFERRED EMBODIMENTS

A method of treating hypertension can include administering to a patient in need thereof a therapeutically effective amount of an extract of Matricaria chamomilla. The extract can be administered orally to the patient in an amount of about 100 mg/kg to about 200 mg/kg. The extract can be prepared using alcohol extraction, water distillation (water distillation method), or water distillation and lyophilization (water distillation and lyophilization method). Preferably, the sample includes aerial parts of the Matricaria chamomilla plant, e.g., leaves and/or flowers.

An alcohol extract of Matricaria chamomilla (Extract 1) can be prepared by obtaining a sample of Matricaria chamomilla, drying the sample, pulverizing the sample to provide a fine powder; and extracting the sample by percolation in alcohol until complete exhaustion to provide a crude alcoholic extract. The sample can include chamomile flowers picked in the flowering stage and dried in the shade and/or air. The “complete exhaustion” can be achieved by percolating four times in four days, e.g., once a day for four days, in this manner. The crude alcoholic extract can be concentrated under reduced pressure to yield a dry or solid extract (Extract 1). The percolation can be carried out for about 72 hours.

An essential oil extract of Matricaria chamomilla (Extract 2) can be prepared by water distillation, e.g., by boiling the Matricaria chamomilla sample and separating essential oils from the heated mixture to provide an aqueous extract including essential oils (Extract 2). The water can then be vaporized using lyophilization to provide a freeze-dried sample. For example, the Matricaria chamomilla sample can be filtered from the water mixture to obtain a filtrate and the filtrate can be lyophilized to provide a dried extract (Extract 3).

Chamomile (Matricaria chamomilla L.) is a medicinal plant species from the Asteraceae family often referred to as the “star among medicinal species.” Chamomile has moderate antioxidant, antimicrobial activities and significant anti-platelet activity in vitro. Animal model studies indicate potent anti-inflammatory action, some anti-mutagenic, cholesterol-lowering activities, as well as anti-spasmodic and anxiolytic effects. Chamomile was found to have the most effective anti-leishmanial activity and its multi-therapeutic, cosmetic, and nutritional values have been established through years of traditional medicinal use as well as through scientific research.

As used herein, a therapeutically effective amount of the extract or an amount effective to treat or prevent hypertension may be determined initially from in vivo studies described herein. For example, an effective amount of the total alcoholic, essential oil or an aqueous extract can be about 100-400 mg/kg, and preferably about 100 mg/kg to about 200 mg/kg.

As used herein, the term “lyophilization” is a means of drying, achieved by freezing the wet substance and causing the ice to sublime directly to vapor by exposing it to a low partial pressure of water vapor without passing through the liquid phase.

The present technology, thus generally described, will be understood more readily by reference to the following examples, which are provided by way of illustration.

### Example 1

### Preparation of Matricaria chamomilla L. Extract

The aerial parts of Chamomile (Matricaria chamomilla L.) (Asteraceae) were collected from the Al-Alnofood desert region of Saudi Arabia during April 2015. The plant sample was air-dried in shade, reduced to fine powder, packed in tightly closed containers, and stored for phytochemical and pharmacological studies. For volatile oil extraction, a separate plant sample was collected in the flowering stage and kept in the refrigerator until needed. The air dried powder of the aerial parts (300 kg) of Matricaria chamomilla L. was extracted by percolation in 1 L of ethanol (95%) until complete exhaustion, by repeating 4 extractions in 4 days. The total ethanol extract was concentrated under reduced pressure at a temperature not exceeding 35° C. to yield a dry extract of 25 g (Extract 1).

### Example 2

### Extraction of the Essential Oils

Essential oils of the plant under investigation (Matricaria chamomilla L.) were extracted using hydro distillation in a Clevenger type apparatus. About 500 g of fresh leaves were placed in a round bottom flask, 1000 ml (1 L) of water was added, and the mixture was boiled for about 5 hours. The essential oils were collected in opaque, small vials. Yields of obtained essential oil were calculated as weight/weight of the sample. Oil samples were stored in a refrigerator for further investigation. Extraction of essential oils produced very low yield (0.5%) and this essential oil was designated as (“Extract 2”). After removal of the volatile oil, the remaining water was filtered off the leaves, which contained the rest of the active material. The filtrate was further concentrated by freeze drying (lyophilization) in order to vaporize the water. The freeze-dried remaining sample was designated as “Extract 3”.

### Example 3

### Determination of Median Lethal Dose (LD50)

Male Sprague-Dawley rats weighing 200-250 g were used for the trials. The rats were housed in standard conditions and fed rodent diet and water ad libitum. The dried Matricaria chamomilla L. extract was freshly suspended in distilled water and then administered to Sprague-Dawley rats of both sexes, (120-140 g) female, and male (150-180 g). The median lethal dose (LD₅₀) of the total alcohol extract was determined by known methods. The Swiss albino mice in groups of six, received one of 500, 1000, 2000, or 5000 mg/kg doses of the tested extract. Control animals received the vehicle and kept under the same conditions. Signs of acute toxicity and number of deaths per dose within 24 h were recorded.

### Example 4

### Effect of Chamomile Extracts on BP and HR of Normotensive Rats

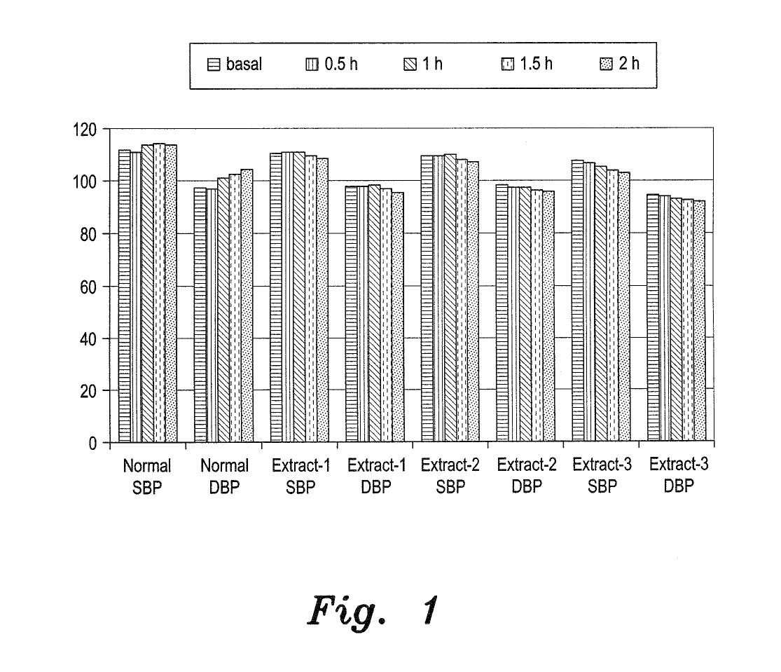

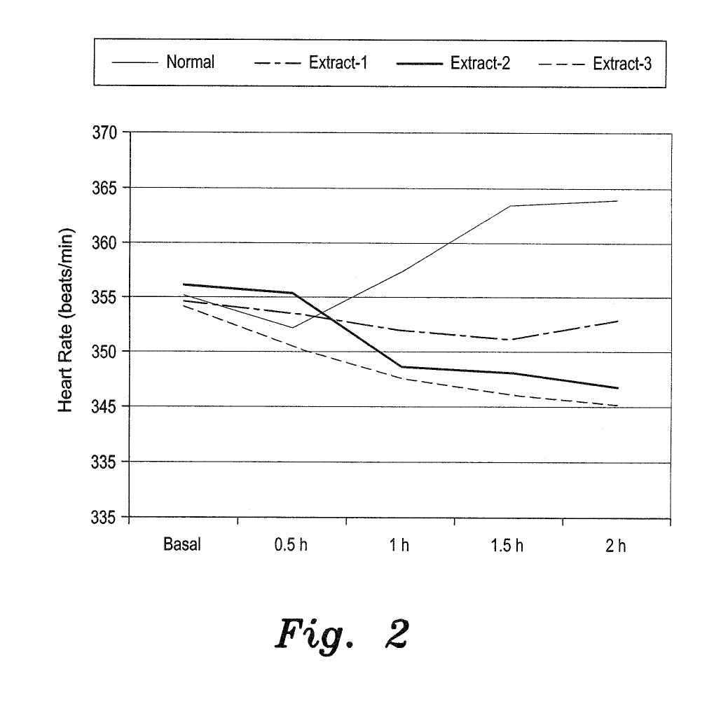

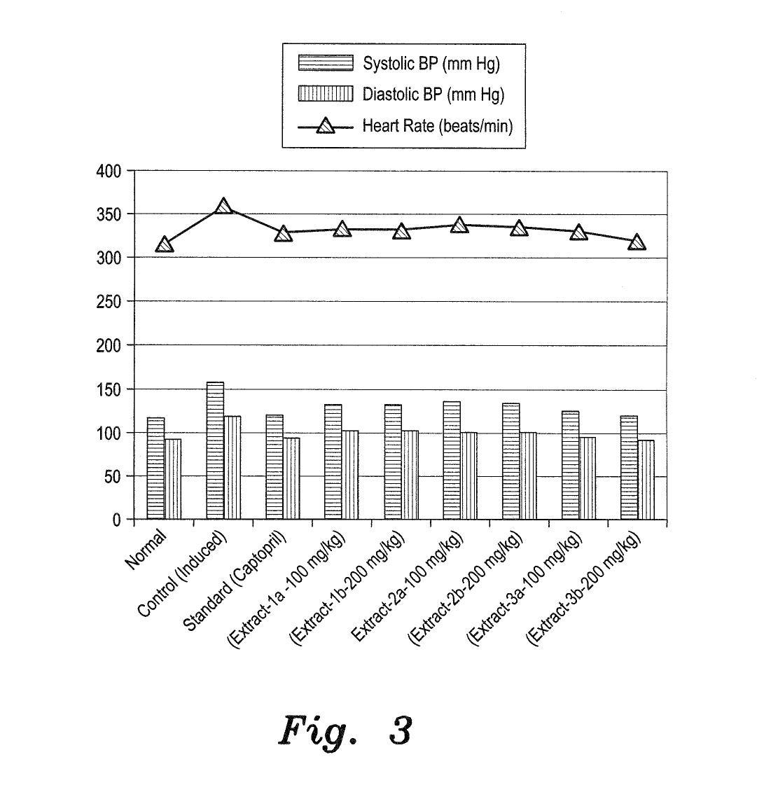

The male Sprague-Dawley rats were divided into four groups of six animals each. Group I (normotensive rats) was administered water, which served as a control group. Group II-Group IV received the plant Extracts 1-3 dissolved in water at a dose of 200 mg/kg in 0.5 mL volume. The effect of the Chamomile extracts on systolic blood pressure, diastolic blood pressure and heart rate were measured by the tail cuff before and after administration at ½ hour intervals up to 2 h (i.e., 0.5 h, 1 h, 1.5 & 2 h respectively). FIG. 1 is a graph showing the effect of a single dose of the Chamomile Extracts 1-3 on blood pressure (BP) of normotensive rats. FIG. 2 is a graph showing the effect of a single dose of the Chamomile Extracts 1-3 on the heart rate (HR) of normotensive rats. FIG. 3 is a graph showing the effect of the three extracts (1-3) on blood pressure (BP) and heart rate (HR) of control and that of hypertension induced rats.

### Example 5

### Induced Hypertension

Male Sprague-Dawley rats were divided into nine groups of six rats each and treated daily for three consecutive weeks. High salt-sucrose solution was given to the rats to induce hypertension in them. Animals of Group I (normal) received tap water (10 mL/kg), Group II (induced control) received salt-sucrose solution (2 ml/100 g p.o., 9% salt solution+10% sucrose solution), Group III (Standard) received salt-sucrose solution (2 ml/100 g p.o., 9% salt solution+10% sucrose solution+captopril 20 mg/kg), Group IV-IX received (2 ml/100 g p.o., 9% salt solution+10% sucrose solution+Plant extract respectively at a dose of 100 mg/kg & 200 mg/kg). At the end of the experimental period, arterial blood pressure and heart rate of all rats were recorded. The rats were sacrificed by decapitation and the free running blood was collected for analysis. The systolic blood pressure (SBP), the diastolic blood pressure (DBP) and the heart rate (HR) of the rats were measured by the tail cuff every day during the entire period of study in control, drug and extract treated animals.

For the estimation of alanine transaminase (ALT), aspartate transaminase (AST), alkaline phosphatase (ALP), total cholesterol, triglycerides, low-density lipoprotein (LDL) and high density lipoprotein (HDL), blood samples were collected in clot activator gel tubes. The serum was separated by centrifuging the blood samples at 5000 rpm for 10 minutes. Serum biochemical parameters were then measured by using commercially available reagent kits.

After collecting the blood samples from the rats, their abdominal cavity was opened; heart, liver and kidney were dissected out and homogenized in Tris-HCl buffer solution to make a 20% homogenate. Glutathione (GSH) and superoxide dismutase (SOD) were determined using the method described by Ellman & Misra for assessing the oxidative damage parameters in some organs.

After induction of hypertension with high salt-sucrose the animals significantly showed an increase in blood pressure. The systolic pressure, diastolic pressure and heart rate were significantly increased when compared to the normal animals. The extract treated groups showed significant reduction in the pressure and heart rate similar to the drug captopril. Captopril inhibits the converting enzyme peptidyldipeptidase that hydrolyzes angiotensin I to angiotensin II and inactivates bradykinin, a potent vasodilator as shown in Tables 1A, B and C. Table 1 A shows the effect of single dose of Chamomile extracts on blood pressure of normotensive rats.

TABLE 1A

| Group | | Basal | 0.5 h | 1 h | 1.5 h | 2 h |
|---|---|---|---|---|---|---|
| Group I | SBP | 111.67 ± 12.27 | 110.54 ± 12.15 | 113.44 ± 10.1 | 114.12 ± 13.8 | 113.39 ± 14.94 |
| (Normal) | DBP | 97.12 ± 8.69 | 96.89 ± 13.83 | 101 ± 9.0.2 | 102.22 ± 11.23 | 104.34 ± 11.47 |
| Group II | SBP | 110.11 ± 10.9 | 110.97 ± 9.33a^{@} | 110.55 ± 10.94a^{@} | 109.47 ± 14.42a^{@} | 108.65 ± 14.32a^{@} |
| (Extract-1 | DBP | 97.51 ± 97.5 | 97.82 ± 9.68a^{@} | 98.13 ± 8.78a^{@} | 96.72 ± 9.57a^{@} | 95.35 ± 8.53a^{@} |
| 200 mg/kg) | | | | | | |
| Group III | SBP | 109.21 ± 14.39 | 109.53 ± 14.43a | 109.61 ± 15.64a^{@} | 108.12 \grave{ } ± 13.1a^{@} | 107.12 ± 10.6a^{@} |
| (Extract-2 | DBP | 97.99 ± 11.8 | 97.21 ± 13.87a^{@} | 97.44 ± 12.84a^{@} | 96.18 ± 11.06a^{@} | 95.81 ± 12.62a^{@} |
| 200 mg/kg) | | | | | | |
| Group IV | SBP | 107.42 ± 9.61 | 106.49 ± 15.19a* | 105.12 ± 11.55a* | 103.88 ± 9.29a* | 102.98 ± 10.19a* |
| (Plant | DBP | 94.71 ± 8.47 | 93.86 ± 9.29a* | 93.09 ± 10.23a* | 92.44 ± 12.18a* | 91.95 ± 12.12a* |
| Extract-3 | | | | | | |
| 200 mg/kg) | | | | | | |

Values are expressed as mean ± SD of 6 animals.
Comparisons were made between:
aGroup I vs. II, III, and IV.
Symbols represent Statistical significance:
*p < 0.01,
^{@}p < 0.05.

Table 1B shows the effect of single dose of Chamomile extracts on HR of normotensive rats.

TABLE 1B

| | Heart rate (beats/min) | | | | |
|---|---|---|---|---|---|
| Group | Basal | 0.5 h | 1 h | 1.5 h | 2 h |
| Group I | 355.22 ± 46.8 | 352.12 ± 39.03 | 357.35 ± 50.99 | 363.49 ± 32.5 | 364 ± 44 |
| (Normal) | | | | | |
| Group II | 354.64 ± 42.9 | 353.56 ± 46.58 a^{@} | 351.92 ± 46.37 a^{@} | 351.12 ± 34.86 a^{@} | 352.89 ± 31.6 a^{@} |
| (Extract-1 200 | | | | | |
| mg/kg) | | | | | |
| Group III | 356.12 ± 43 | 355.41 ± 50.71 a^{@} | 348.66 ± 45.94 a^{@} | 348.12 ± 34.46 a^{@} | 346.76 ± 41.9 a^{@} |
| (Extract-2 | | | | | |
| 200 mg/kg) | | | | | |
| Group IV | 354.13 ± 31.7 | 350.45 ± 34.69 a* | 347.56 ± 49.59 a* | 346.12 ± 38.06 a* | 345.19 ± 37.94 a* |
| (Plant Extract- | | | | | |
| 3 200 mg/kg) | | | | | |

Values are expressed as mean ± SD of 6 animals.
Comparisons were made between: a-Group I vs II, III, IV.
Symbols represent Statistical significance.
*-p < 0.01,
^{@}-p < 0.05.

Table 1C shows the effect of Extracts on Blood Pressure and Heart Rate of Control and hypertension Induced Rats.

TABLE 1C

| | Systolic BP | Diastolic BP | Heart rate |
|---|---|---|---|
| Groups | (mm Hg) | (mm Hg) | (beats/min) |
| Group I (Normal) | 118.28 ± 16.88 | 92.64 ± 12.21 | 316.31 ± 41.68 |
| Group II | 158.34 ± 17.4 | 119.71 ± 13.61 | 359.42 ± 51.29 |
| (Induced) | | | |
| Group III | 121.45 ± 10.9a* | 95.56 ± 10.5a* | 328.86 ± 36.14a* |
| (Captopril) | | | |
| Group IV | 134.53 ± 16.3a^{@} | 104.39 ± 13.75a^{@} | 334.54 ± 36.77a^{@} |
| (Extract-1a-100 | | | |
| mg/kg) | | | |
| Group V | 133.18 ± 14.64 a^{@} | 103.32 ± 10.23a^{@} | 332.77 ± 36.57a^{@} |
| (Extract-1b-200 | | | |
| mg/kg | | | |
| Group VI | 137.67 ± 15.13 a^{@} | 102.44 ± 12.4 a^{@} | 339.66 ± 48.47 a^{@} |
| (Extract-2a-100 | | | |
| mg/kg) | | | |
| Group VII | 135.41 ± 17.71 a^{@} | 100.93 ± 9.03 a^{@} | 335.87 ± 40.6 a^{@} |
| (extract-2b-200 | | | |
| mg/kg) | | | |
| Group VIII | 126.75 ± 15.3 a* | 96.15 ± 10.57a* | 330.91 ± 40a* |
| (Extract-3a-100 | | | |
| mg/kg) | | | |
| Group IX | 120.82 ± 13.83a* | 92.27 ± 10.36a* | 320.45 ± 47.01a* |
| (Extract-3b-200 | | | |
| mg/kg) | | | |

Values are expressed as mean ± SD of 6 animals.
Comparisons were made between: a-Group II vs HI, IV, V& VI.
Symbols represent Statistical significance.
*-p < 0.01,
^{@}-p < 0.05.

The alteration in the level of serum marker enzymes SGOT, SGPT, ALP and Lipid profile is shown in Table 2 & 3 respectively. Increased levels of AST, ALT, ALP and alternation in the lipid profile were brought back significantly to near normal by the treatment with captopril and plant extracts. The effect of extracts on serum marker enzymes of control and hypertension induced rats is shown in Table 2.

TABLE 2

| | AST | ALT | ALP |
|---|---|---|---|
| Groups | (IU/L) | (IU/L) | (IU/L) |
| Group I (Normal) | 41.25 ± 4.98 | 82.62 ± 10.89 | 128.24 ± 15.5 |
| Group II (Induced) | 69.48 ± 7.63 | 112.93 ± 14.88 | 163.18 ± 17.93 |
| Group III | 45.03 ± 5.44a* | 85.44 ± 10.33a* | 132.12 ± 17.41a* |
| (Captopril) | | | |
| Group IV | 53.72 ± 7.08a^{@} | 96.76 ± 10.63a^{@} | 141.25 ± 15.52a^{@} |
| (Extract-1a-100 | | | |
| mg/kg) | | | |
| Group V | 52.44 ± 6.9a^{@} | 95.18 ± 8.51a^{@} | 140.36 ± 13.89 a^{@} |
| Extract-1b 200 | | | |
| mg/kg | | | |
| Group VI | 56.83 ± 6.24 a^{@} | 99.23 ± 12 a^{@} | 144.87 ± 15.92a^{@} |
| (Extract-2a-100 | | | |
| mg/kg) | | | |
| Group VII | 55.72 ± 6.124a^{@} | 98.92 ± 13.03a^{@} | 142.41 ± 17.2a^{@} |
| (Extract-2b-200 | | | |
| mg/kg) | | | |
| Group VIII | 50.44 ± 6.09a* | 92.54 ± 8.28a* | 141.46 ± 12.7a* |
| (Extract-3a-100 | | | |
| mg/kg) | | | |
| Group IX | 49.36 ± 5.425a* | 91.37 ± 11a* | 136.45 ± 12.2a* |
| (Extract-3b-200 | | | |
| mg/kg) | | | |

Values are expressed as mean ± SD of 6 animals.
Comparisons were made between: a-Group II vs III, IV, V& VI.
Symbols represent Statistical significance.
*-p < 0.01,
^{@}-p < 0.05

The effect of extracts on plasma lipid profile of control and hypertension induced rats are shown in Table 3.

TABLE 3

| | CHOLESTEROL | TRIGLYCERIDES | LDL | HDL |
|---|---|---|---|---|
| Groups | (mg/dL) | (mg/dL) | (mg/dL) | (mg/dL) |
| Group I (Normal) | 128.46 ± 18.33 | 91.16 ± 9.04 | 76.43 ± 8.4 | 45.71 ± 5.02 |
| Group II (Induced) | 172.57 ± 20.9 | 169.84 ± 18.67 | 128.75 ± 16.96 | 28.87 ± 2.85 |
| Group III | 135.42 ± 13.41a* | 106.41 ± 9.52a* | 89.23 ± 9.8a* | 41.46 ± 4.55a* |
| (Captopril) | | | | |
| Group IV | 145.74 ± 14.43 a^{@} | 116.43 ± 10.4 a^{@} | 97.29 ± 10.69 a^{@} | 34.09 ± 3.74 a^{@} |
| (Extract-1a-100 | | | | |
| mg/kg) | | | | |
| Group V | 142.79 ± 17.3a^{@} | 112.93 ± 12.41a^{@} | 91.68 ± 9.07a^{@} | 37.06 ± 3.31a^{@} |
| (Extract-1b-200 | | | | |
| mg/kg | | | | |
| Group VI | 152.34 ± 16.74a^{@} | 120.65 ± 13.26a^{@} | 99.32 ± 13.09a^{@} | 37.86 ± 4.98a^{@} |
| (Extract 2a-100 | | | | |
| mg/kg) | | | | |
| Group VII | 150.27 ± 14.88a^{@} | 117.64 ± 12.93a^{@} | 97.25 ± 10.69a^{@} | 38.85 ± 4.69a^{@} |
| (extract 2b-00 | | | | |
| mg/kg) | | | | |
| Group VIII | 140.81 ± 12.6 a* | 106.11 ± 12.8a* | 87.22 ± 8.63a* | 35.19 ± 3.15a* |
| (Extract-3a-100 | | | | |
| mg/kg) | | | | |
| Group IX | 138.94 ± 12.4a* | 102.35 ± 9.15a* | 84.29 ± 8.34a* | 40.42 ± 3.62a* |
| (Extract-3b-200 | | | | |
| mg/kg) | | | | |

Values are expressed as mean ± SD of 6 animals.
Comparisons were made between: a-Group II vs. III, IV, and V & VI.
Symbols represent statistical significance.
*-p < 0.01,
^{@}-p < 0.05.

### Example 6

### ACE Inhibition Assay

Human blood serum was used as angiotensin-converting enzyme (ACE) source. The determination of the ACE activity in serum was carried out in vitro by using the method by Simonetta Ronca-Testoni modified by the Biochemistry Laboratory at Universidad del Quindio, plus some considerations proposed by Serra et al. (2005) for an ACE inhibition assay using plant extracts. The method is based on enzymatic hydrolysis of the Furilacriloil-L-phenylalanyl-glycyl-glycine (FAPGG), by the serum ACE, to Furilacriloil-L-phenyl(FAP) and glycyl-glycine(Gly-Gly). Briefly, two tubes with 2.5 ml of serum each received the addition of 22.5 ml of distilled water, 25.0 ml buffer (0.8 mM FAPGG, 400 mM NaCl, 50 mM HEPES pH 8.2), and plant extract at a concentration of 0.1 mg/ml in reaction mixture. As a blank, another tube was used containing exactly the same, plus EDTA 3.3 mM as an ACE inhibitor. Distilled water was used as a negative control and (80 nmol/l), as positive control. The tubes were incubated at 37° C. for 20 minutes and left to stand on ice to halt the enzymatic reaction. Finally, absorbance was read for each at 345 nm by using a Milton Roy Genesis 5 spectrophotometer. Five assays were performed per extract, one with each serum sample. During each assay, ACE activity with plant extract was measured in triplicate. The activity was obtained by applying the following equation:

$$ACE activity = \frac{( Δ A \times Vfx \times \frac{1000}{t} )}{( 0.5 \times Vs )} \qquad ( 1 )$$

Where ΔA is the absorbance difference between the samples and the blank, Vfx the test final volume, 1000 converts ml into liter, t is incubation time, 0.5 is the hydrolysis absorbance of 1 mM of FAPGG under test conditions, and Vs is the volume of the serum sample (0.025 ml). ACE activity is expressed in ACE Units per liter (U/L). An ACE unit (1 U) is the amount of the enzyme that converts 1 mmol of FAPGG in FAP and Gly-Gly per minute at 37° C.

The percentage of inhibition (% I) of each extract on ACE was determined by using the equation:

$$\% I = \frac{[ ( Ac - As ) ]}{Ac} \times 100 \qquad ( 2 )$$

Where Ac is ACE activity for negative control and As is the ACE activity in the presence of the plant extract or Captopril. Values are expressed as the average of the inhibition obtained in the six repetitions.

The effect of extracts on angiotensin-converting enzyme activity is shown in Table 4. As shown in this Table 4, an increase in the Angiotensin-converting enzyme activity is characteristically seen in induced group, which is significantly reduced in both extract treated and captopril treated animals.

TABLE 4

| | ACE ACTIVITY |
|---|---|
| Groups | (U/L) |
| Group I (Normal) | 36.92 ± 3.3 |
| Group II (Induced) | 83.52 ± 7.42 |
| Group III (Captopril) | 38.06 ± 3.76a* |
| Group IV (Extract-1a-100 mg/kg) | 55.28 ± 5.47a^{@} |
| Group V (Extract-1b-200 mg/kg} | 52.87 ± 5.23 a^{@} |
| Group VI (Extract-2a-100 mg/kg) | 53.87 ± 5.33a^{@} |
| Group VII (extract-2b-200 mg/kg) | 51.44 ± 4.6 a^{@} |
| Group VIII (Extract-3a-100 mg/kg) | 44.81 ± 4.92 a* |
| Group IX (Extract-3b-200 mg/kg) | 41.31 ± 3.69 a* |

Values are expressed as mean ± SD of 6 animals.
Comparisons were made between: a-Group II vs III, IV, V& VI.
Symbols represent Statistical significance.
*-p < 0.01,
^{@}-p < 0.05.

Similarly the change in the levels of enzymic antioxidants is established and retained to near normal values significantly in the captopril and extract treated groups and is shown in Table 5. Table 5 shows the effect of extracts on tissue homogenates of control and hypertension induced rats.

TABLE 5

| | Liver GSH | SOD | Heart GSH | SOD | Kidney GSH | SOD |
|---|---|---|---|---|---|---|
| Groups | (mM/L) | (U/mg) | (mM/L) | (U/mg) | (mM/L) | (U/mg) |
| Group I | 25.08 ± 2.48 | 67.49 ± 8.15 | 34.87 ± 4.59 | 88.45 ± 7.91 | 16.59 ± 1.82 | 74.23 ± 7.15 |
| (Normal) | | | | | | |
| Group II | 16.27 ± 1.78 | 26.37 ± 3.47 | 22.39 ± 2.71 | 34.32 ± 4.15 | 11.31 ± 1.24 | 32.12 ± 3.53 |
| (Induced) | | | | | | |
| Group III | 28.92 ± 4.12a | 42.67 ± 5.62a | 38.78 ± 4.69a | 68.92 ± 9.08a^{@} | 31.04 ± 2.78a^{@} | 67.96 ± 6.08a^{@} |
| (Captopril) | | | | | | |
| Group IV | 19.16 ± 1.71a^{@} | 39.84 ± 4.81a^{@} | 24.02 ± 2.37a^{@} | 56.14 ± 6.17a^{@} | 17.68 ± 1.94a^{@} | 39.24 ± 4.74a^{@} |
| (Extract-1 - | | | | | | |
| 100 mg/kg) | | | | | | |
| Group V | 20.23 ± 1.81a^{@} | 41.12 ± 4.07a^{@} | 26.81 ± 2.94a^{@} | 51.31 ± 6.2a^{@} | 33.87 ± 4.46a^{@} | 40.41 ± 4.88a^{@} |
| (Extract- 1b- | | | | | | |
| 200 mg/kg | | | | | | |
| Group VI | 18.66 ± 2.05a^{@} | 39.27 ± 3.51a^{@} | 28.56 ± 4.07a^{@} | 53.43 ± 6.46a^{@} | 32.19 ± 3.89a^{@} | 39.55 ± 3.91a^{@} |
| (Extract- 2a- | | | | | | |
| 100 mg/kg) | | | | | | |
| Group VII | 21.45 ± 2.12a^{@} | 46.52 ± 5.11a^{@} | 29.23 ± 2.61a^{@} | 36.04 ± 4.05a^{@} | 14.86 ± 1.47a^{@} | 43.61 ± 4.79a^{@} |
| (extract- 2b- | | | | | | |
| 200 mg/kg) | | | | | | |
| Group VIII | 24.83 ± 2.22a* | 58.14 ± 8.29a* | 42.54 ± 5.14a* | 53.76 ± 4.81a* | 29.72 ± 3.91a* | 49.26 ± 7.09a* |
| (Extract- 3a- | | | | | | |
| 100 mg/kg) | | | | | | |
| Group IX | 22.17 ± 2.68a^{@} | 41.27 ± 4.99a^{@} | 24.13 ± 2.38a^{@} | 51.29 ± 4.59a^{@} | 12.31 ± 1.1a^{@} | 40.61 ± 4.02a^{@} |
| (Extract- 3b - | | | | | | |
| 200 mg/kg) | | | | | | |

Values are expressed as mean ± SD of 6 animals.
Comparisons were made between:
aGroup II vs III, IV, V& VI.
Symbols represent Statistical significance.
*p < 0.01,
^{@}p < 0.05.

### Example 7

### Clinical Applications

Chamomile was administered to a number of patients with different types of blood pressure values, some were chronic hypertensive patients and others were normal patients. Blood pressure was measured every half an hour after giving the patient two tablespoons or 3 tablespoons of chamomile after decoction in hot water. This study was designed to explore the effects of oral administration of chamomile on BP and HR in human volunteers. The subjects were chosen as follows. Four hundred independent-living nonsmoking men (n=200) and women (n=200) in Riyadh (range 22-40 years and body weight 65-105 kg) were recruited to take part in a clinical trial of the effects of chamomile drinking on blood pressure. They were either normotensive (SBP of 120-124 mmHg and/or DBP of 70-86 mmHg) or mildly hypertensive (SBP of 139-159 mm Hg) and/or DBP of 86-99 mm Hg). Written informed consent was obtained from all participants after they received a full written explanation of the content and the aim of the study.

The study protocol was as follows. The ratios of normotensive to mild hypertensive males and females in all groups were intended to be 1:1. The subjects were given the following instructions: continue usual dietary habits without drinking and eating too much: and no change in exercise habits. In addition, subjects were prohibited from ingesting blood pressure-influencing drugs. On the day before examination, they were instructed to finish dinner by 9:00 p.m. and not to eat or drink anything but water until the end of examination. On the day of examination, BP and HR were measured as baseline with a strain-gauge plethysmograph after the subject arrived at the clinic and sat quietly for at least 10 min. Each volunteer received a single cup of chamomile beverage (250 mL), between 8:00 and 10:00 a.m. Chamomile beverage was made with just boiled water with two teaspoonful of the plant flower and this beverage was consumed within 10 min. BP (SBP and DBP) and HR of each subject were measured at; 0.0, 0.5, 1.0, 1.5 and 2.0 h intervals post-administration. All measurements were performed for subjects in the supine position in a temperature-controlled (24° C. to 27° C.), quiet, dark laboratory. The results are expressed as; mean±SE. Values of p<0.05 and p<0.05 were considered to indicate statistical significance. The effects of interventions on BP and HR were analyzed with the paired Student's t-test.

The effect of Chamomile beverages on BP and HR of normotensive and mild hypertensive human volunteers were studied. One hour following oral administration of a cup of chamomile to normotensive human volunteers, the mean SBP detected were 98.7 mmHg, respectively, compared to their basal values 120.1 mmHg (p<0.05). The highest hypotensive activity was recorded 1.5 h post administration as shown in Table 6 (p<0.05) below. The same beverages significantly reduced the DBP (65.2 mmHg) and HR70b/mint (Table 6) 1.5 h after their oral administration to normotensive human volunteers.

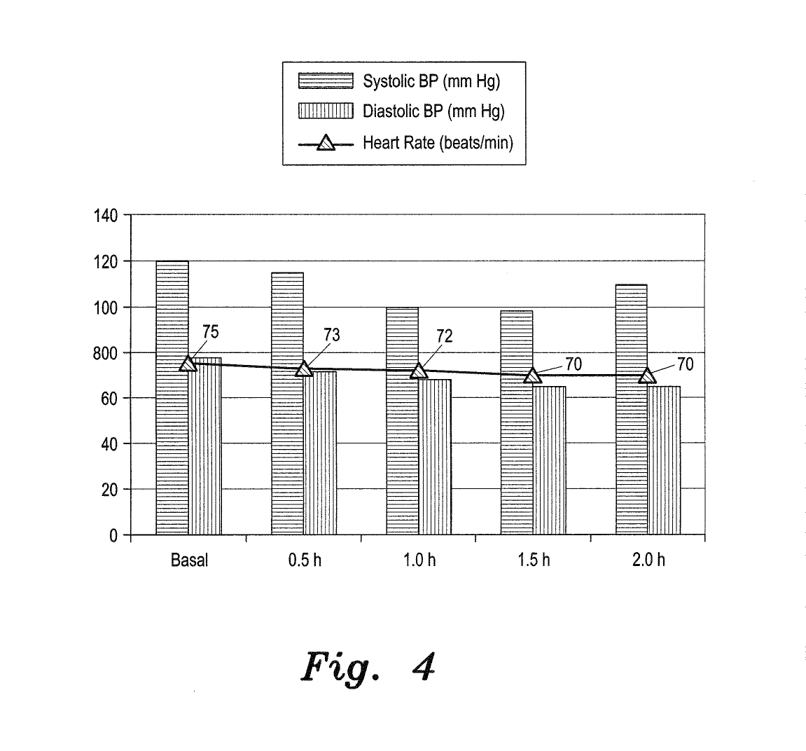

Oral administration of a cup of Chamomile with one, two and three teaspoonful of plant powder to mildly hypertensive human volunteers, significantly decreased SBP, DBP and HR (Table 7, 8 & 9), 1.5 h post-administration, compared with their basal values (p<0.05). Typically, 1 teaspoonful is ˜5 g of chamomile dried flower. The hypertensive effects were varied according to the number of teaspoonful which were used, the highest effect was recorded with 3 teaspoonfuls for longer duration. FIG. 4 is a graph showing the effect of oral administration of a single cup (250 mL) of chamomile beverage on the SBP and DBP & heart rate in normotensive human volunteers (n=50).

The effect of oral administration of a single cup (250 mL) of chamomile beverage on the SBP and DBP & heart rate in normotensive human volunteers (n=50) is provided in Table 6.

TABLE 6
Systolic and diastolic blood pressure (mmHg) after

| Pressure | | | | | |
|---|---|---|---|---|---|
| & HR | Basal | 0.5 h | 1.0 h | 1.5 h | 2.0 h |
| SBP | 120.1 ± 2.80 | 115.3 ± 1.14 | 100.1 ± 2.6 | 98.7 ± 2.13 ^{a} | 110.10 ± 1.21 |
| DBP | 78.10 ± 1.51 | 73.1 ± 1.61 | 68.3 ± 3.12 | 65.2 ± 1.82 ^{a} | 65.20 ± 1.56 |

Heart Rate (beats/min)

| | | | | | |
|---|---|---|---|---|---|
| Heart rate | 75 ± 1.29 | 73 ± 2.18 | 72 ± 1.18 | 70 ± 1.65 ^{a} | 70 ± 3.16 |

Values represent the mean ± SE.
^{a}p < 0.05.

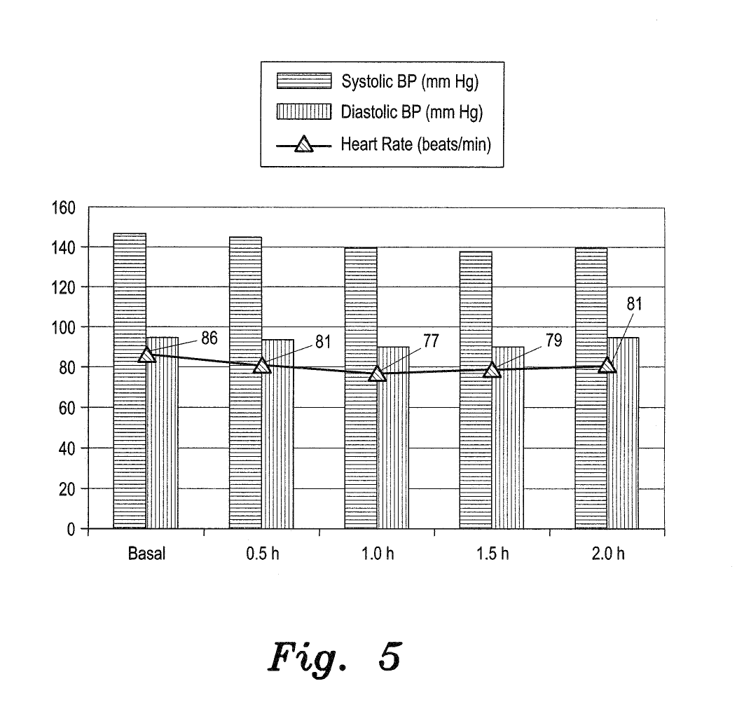

Table 7 shows the effect of oral administration of a single cup (250 mL) with one teaspoonful of chamomile flower on the SBP and DBP in mildly hypertensive human volunteers (n=50). FIG. 5 illustrates the effect of oral administration of a single cup (250 mL) of chamomile beverage on the SBP and DBP in mildly hypertensive human volunteers.

TABLE 7
Systolic and diastolic blood pressure (mmHg)

| Pressure & HR | Basal | 0.5 h | 1.0 h | 1.5 h | 2.0 h |
|---|---|---|---|---|---|
| SBP | 147.1 ± 2.15 | 145.2 ± 2.11 | 140.1 ± 3.12 | 138.1 ± 1.22^{a} | 140.1 ± 2.10 |
| DBP | 95.2 ± 1.35 | 93.7 ± 1.18 | 90.2 ± 3.15 | 90.3 ± 1.49^{a} | 952 ± 1.25 |

Heart Rate (beats/min)

| | | | | | |
|---|---|---|---|---|---|
| Heart rate | 86 ± 2.25 | 81 ± 2.25 | 77 ± 2.26^{a} | 79 ± 2.74 | 81 ± 2.23 |

Values represent the mean ± SE.
^{a}p < 0.05.

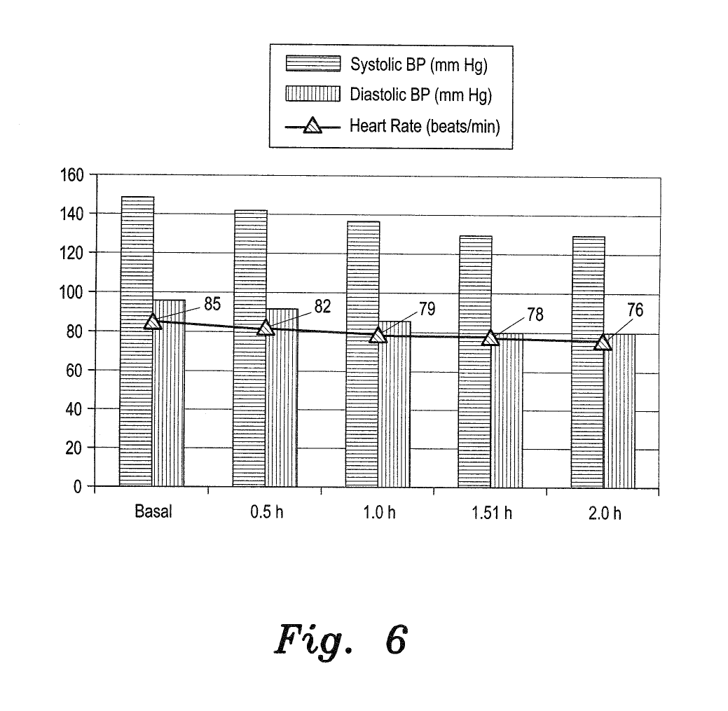

Table 8 shows the effect of oral administration of a single cup (250 mL) with two teaspoons of chamomile flower on the SBP and DBP in in mildly hypertensive human volunteers (n=50). FIG. 6 is a graph illustrating the effect of oral administration of a single cup (250 mL) with two teaspoons (2×˜5 g) of chamomile flower on the SBP and DBP in mildly hypertensive human volunteers.

TABLE 8
Systolic and diastolic blood pressure (mmHg)

| Pressure & HR | Basal | 0.5 h | 1.0 h | 1.5 h | 2.0 h |
|---|---|---|---|---|---|
| SBP | 149.1 ± 2.15 | 142.2 ± 2.11 | 137.1 ± 3.12 | 130.1 ± 1.22^{a} | 130.1 ± 2.10^{a} |
| DBP | 96.2 ± 1.22 | 92.1 ± 1.18 | 86.3 ± 3.15 | 80.2 ± 1.49^{a} | 80.1 ± 1.25^{a} |

Heart Rate (beats/min)

| | | | | | |
|---|---|---|---|---|---|
| Heart rate | 85 ± 1.23 | 82 ± 3.28 | 79 ± 1.25 | 78 ± 1.44 | 76 ± 1.13^{a} |

Values represent the mean ± SE.
^{a}p < 0.05.

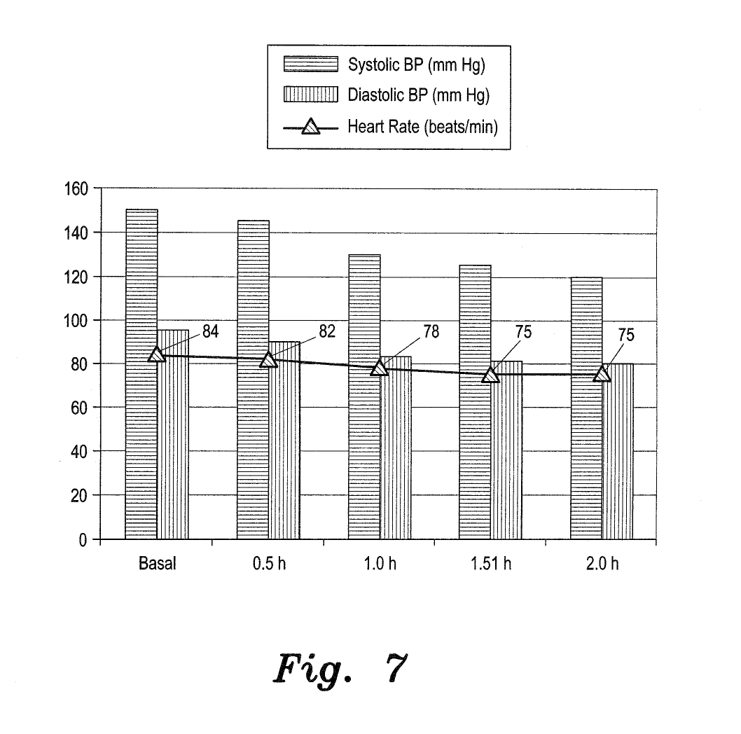

Table 9 shows the effect of oral administration of a single cup (250 mL) with Three teaspoonful of chamomile flower on the SBP and DBP in in mildly hypertensive human volunteers (n=50). FIG. 7 illustrates the effect of oral administration of a single cup (250 mL) with three teaspoonful (3×˜5 g) of chamomile flower on the SBP and DBP in mildly hypertensive human volunteers (n=50).

TABLE 9
Systolic and diastolic blood pressure (mmHg)

| Pressure & HR | Basal | 0.5 h | 1.0 h | 1.5 h | 2.0 h |
|---|---|---|---|---|---|
| SBP | 150.1 ± 1.15 | 145.4 ± 1.12 | 130.3 ± 1.12 | 125.4 ± 1.23 | 120.1 ± 1.12^{a} |
| DBP | 95.3 ± 2.12 | 90.1 ± 1.18 | 83.3 ± 3.15 | 81.2 ± 1.49 | 80.1 ± 1.25^{a} |

Heart Rate (beats/min)

| | | | | | |
|---|---|---|---|---|---|
| Heart rate | 84 ± 1.21 | 82 ± 2.21 | 78 ± 1.20 | 75 ± 1.54 | 75 ± 1.19^{a} |

Values represent the mean ± SE.
^{a}p < 0.05.

The three extracts were tested for activities as antihypertensive agent on normotensive and hypertensive rats. From the results it can be concluded that upon testing the different plant extracts of Chamomile (Matricaria chamomilla L.) (“Extracts 1-3”) on laboratory animals, all three of the extracts exhibited anti-hypertensive activity to greater extent in developed hypertension in rats. The activity of the extracts was more predominant in Plant extract 3 than Plant extracts 1& 2. The plant extracts showed no side effects on liver, heart and kidney functions. Clinical application on human volunteers using Chamomile beverage using different concentration (1, 2 and 3 teaspoonful) showed very good anti-hypertensive activity on both normal and mildly hypertensive human. The magnitude of response produced by the Chamomile beverages was higher in mildly hypertensive than in normotensive volunteers and was dose dependent, i.e., the more the number of teaspoonfuls used, the higher the anti-hypertensive activities produced (3>2>1 teaspoonful/250 ml)). The total alcohol extract was found to be safe up to 4000 mg/kg, and there were no side effects reported on liver and kidney functions. While it is known that Chamomile contains flavonoids, volatile oils, terpenes, coumarins and tannins, it can be surmised that flavonoids present in the flowers of Matricaria chamomilla L. may be responsible for the lowering of the blood pressure.

It is to be understood that the present invention is not limited to the embodiments described above, but encompasses any and all embodiments within the scope of the following claims.

1. A method of treating hypertension, comprising administering orally to a patient in need thereof a therapeutically effective amount of an extract of Matricaria chamomilla only, wherein the Matricaria chamomilla is from the Al-Alnofood desert region of Saudi Arabia, further wherein the therapeutically effective amount of the extract of Matricaria chamomilla is about 100 mg/kg to about 200 mg/kg.

2. (canceled)

3. (canceled)

4. The method of treating hypertension according to claim 1, wherein the extract of Matricaria chamomilla is prepared by:
providing a sample derived from aerial parts of Matricaria chamomilla,
drying the sample,
pulverizing the sample to provide a fine powder; and
extracting the powder by alcohol extraction.

5. The method of treating hypertension according to claim 4, wherein the sample is obtained from flowers of Matricaria chamomilla.

6. The method of treating hypertension according to claim 4, wherein the alcohol extraction comprises:
percolating the fine powder in alcohol to provide a crude alcoholic extract;
filtering the extract to obtain a crude alcoholic extract; and
concentrating the crude alcoholic extract to obtain a solid extract.

7. The method of treating hypertension according to claim 6, wherein the percolation is carried out for about 72 hours.

8. The method of treating hypertension according to claim 1, wherein the extract of Matricaria chamomilla is prepared by water distillation, the water distillation comprising boiling the Matricaria chamomilla to provide a heated mixture and separating essential oils from the heated mixture to provide an aqueous extract including essential oils.

9. The method of treating hypertension according to claim 8, wherein the water distillation method further comprises vaporizing water from the heated mixture using lyophilization to provide a dried extract.

10. The method of treating hypertension according to claim 8, wherein the extract is derived from aerial parts of Matricaria chamomilla.

11. A method of preparing an extract of Matricaria chamomilla, comprising:
providing a sample derived from aerial parts of Matricaria chamomilla,
drying the sample,
pulverizing the sample to provide a fine powder; and
extracting the powder by alcohol extraction.

12. The method of preparing an extract of Matricaria chamomilla according to claim 11, wherein the sample is obtained from flowers of Matricaria chamomilla.

13. The method of preparing an extract of Matricaria chamomilla according to claim 11, wherein the alcohol extraction comprises:
percolating the fine powder in alcohol to provide a crude alcoholic extract;
filtering the extract to obtain a crude alcoholic extract; and
concentrating the crude alcoholic extract to obtain a solid extract.

14. The method of preparing an extract of Matricaria chamomilla according to claim 13, wherein the percolation is carried out for about 72 hours.

15. A method of preparing an extract of Matricaria chamomilla, comprising, water distillation of the sample, the water distillation including boiling the Matricaria chamomilla sample to provide a heated mixture and separating essential oils from the heated mixture to provide an aqueous extract including essential oils.

16. The method of preparing an extract of Matricaria chamomilla according to claim 15, wherein the water distillation method further comprises vaporizing water from the heated mixture using lyophilization to provide a dried extract.
